(19) United States
(12) Patent Application Publication
(10) Pub. No.: US 2026/0191664 A1
(43) Pub. Date: Jul. 9, 2026

(54) BOUNDARY ASSESSMENT INSTRUMENT AND METHOD OF USING THE SAME IN AN ORTHOPAEDIC SURGICAL KNEE PROCEDURE

(71) Applicant: DePuy Ireland Unlimited Company, Ringaskiddy (IE)

(72) Inventors: Michael J. Rock, Leeds (GB); Dustin N. Albert, Warsaw, IN (US); Ali Hussien M. Ersad, Warsaw, IN (US); James Brooks, Burley in Wharfedale (GB)

(21) Appl. No.: 19/424,775

(22) Filed: Dec. 18, 2025

Related U.S. Application Data
(60) Provisional application No. 63/932,072, filed on Dec. 5, 2025.
(60) Provisional application No. 63/741,602, filed on Jan. 3, 2025.

(51) Int. Cl. A61F 2/46

(52) U.S. Cl. CPC A61F 2/461; A61F 2/4657; A61F 2002/4615; A61F 2002/4668

(57) ABSTRACT

A boundary assessment instrument includes a spacer block, a tibial alignment rod that aligns with the patient’s ankle, and a femoral alignment rod that aligns with a patient’s hip. A varus/valgus angle gauge displays the varus/valgus angle of the installed spacer block. A hip-knee-ankle angle gauge displays the overall hip-knee-ankle angle of the patient’s leg created by the installed spacer block. A method of surgically preparing a patient’s femur and tibia is also disclosed.

This application claims priority to U.S. Provisional Patent Application Serial No. 63/741,602 which was filed on January 3, 2025, and U.S. Provisional Patent Application Serial No. 63/932,072 which was filed on December 5, 2025, each of which are hereby incorporated by reference in their entirety.

### TECHNICAL FIELD

The present disclosure relates generally to orthopaedic surgical instruments and, more particularly, to surgical instruments used during an orthopaedic surgical knee procedure.

### BACKGROUND

Joint arthroplasty is a well-known surgical procedure by which a diseased and/or damaged natural joint is replaced by a prosthetic joint. For example, in a total knee arthroplasty surgical procedure, a patient’s natural knee joint is partially or totally replaced by a prosthetic knee joint or knee prosthesis. To facilitate the replacement of the natural joint with the prosthesis, orthopaedic surgeons use a variety of orthopaedic surgical instruments such as, for example, saws, drills, reamers, rasps, broaches, cutting blocks, drill guides, milling guides, and other surgical instruments.

In total knee arthroplasty (TKA), the femur and tibia of the patient’s knee are resected to create planar surfaces onto which a prosthetic femoral component and tibial component, respectively, are installed. Traditional TKA involves determining the resection planes based on a pre-determined angle as a function of mechanical alignment or by using a balanced approach that sets the resection planes based on ligament tension. More recently, kinematic alignment techniques involve determining the resection planes as a function of the native, pre-disease state of the patient’s knee.

During a knee procedure involving either technique, a surgeon may desire to maintain a certain anatomic realignment or outcome. For example, a surgeon may preoperatively plan to restore a patient’s native hip-knee-ankle angle. Similarly, the surgeon may plan to surgically produce a preplanned varus/valgus angle in the patient’s knee. In addition, to preoperatively-planned outcomes, a surgeon may also establish certain boundaries or “maximums” as it relates to the patient’s hip-knee-ankle angle or the varus/valgus angle of the patient’s knee.

### SUMMARY

According to one aspect of the disclosure, an orthopaedic surgical instrument assembly for use during an orthopaedic knee replacement procedure includes an elongated base having a posterior end configured to be positioned between a femur and a tibia of a patient and a shaft extending anteriorly away from the posterior end. The orthopaedic surgical instrument assembly also includes a femoral alignment tool extending superiorly away from the shaft. The femoral alignment tool has (i) a femoral alignment arm rotatably coupled to the shaft, (ii) a hip-knee-ankle arm segment located superior to the shaft, (iii) a hip-knee-ankle pointer located superior to the shaft, and (iv) a hip-knee-ankle angle gauge attached to the hip-knee-ankle arm segment. The orthopaedic surgical instrument assembly also includes a tibial alignment tool extending inferiorly away from the shaft. The tibial alignment tool has (i) a tibial alignment arm rotatably coupled to the shaft, (ii) a varus/valgus arm segment located inferior to the shaft, (iii) a varus/valgus pointer located inferior to the shaft, and (iv) a varus/valgus angle gauge attached to the varus/valgus arm segment. One of the varus/valgus pointer and the varus/valgus angle gauge is in a fixed orientation relative to the elongated base and the other of the varus/valgus pointer and the varus/valgus angle gauge is configured to rotate with the tibial alignment arm. One of the hip-knee-ankle pointer and the hip-knee-ankle angle gauge is in a fixed orientation relative to the tibial alignment arm and configured to rotate with the tibial alignment arm and the other of the hip-knee-ankle pointer and the hip-knee-ankle angle gauge is configured to rotate with the femoral alignment arm.

In an example, the elongated base comprises a spacer block configured to be positioned between a resected femur and a resected tibia of the patient. The spacer block may have a flat superior surface and a flat inferior surface. The shaft may extend anteriorly away from the spacer block.

In another example, a longitudinal axis of the varus/valgus pointer is orthogonal to the flat superior surface of the spacer block.

In an example, a femoral alignment rod extends superiorly away from a superior end of the femoral alignment arm, and a tibial alignment rod extends inferiorly away from an inferior end of the tibial alignment arm. The femoral alignment rod may be extendable and retractable relative to the superior end of the femoral alignment arm, and the tibial alignment rod may be extendable and retractable relative to the interior end of the tibial alignment arm.

In another example, a longitudinal axis of the femoral alignment arm is colinear with a longitudinal axis of the hip-ankle-knee arm segment.

The varus/valgus angle gauge may include a plurality of angle indicators each of which corresponds to a different varus/valgus angle. Rotation of the tibial alignment arm may cause the varus/valgus angle gauge to be moved relative to the varus/valgus pointer such that the varus/valgus pointer aligns with one of the plurality of angle indicators.

The femoral alignment arm and the tibial alignment arm may form an anteroposterior angle, and the anteroposterior angle may be between 155 degrees to 175 degrees.

The hip-knee-ankle angle gauge may include a plurality of angle indicators each of which corresponds to a different hip-knee-ankle angle. Rotation of the tibial alignment arm may cause the hip-knee-ankle angle gauge to be moved relative to the hip-knee-ankle pointer such that the hip-knee-ankle pointer aligns with one of the plurality of angle indicators. Rotation of the femoral alignment arm may cause the hip-knee-ankle pointer to be moved relative to the hip-knee-ankle angle gauge such that the hip-knee-ankle pointer aligns with one of the plurality of angle indicators.

In another example, the femoral alignment arm and the tibial alignment arm may rotate relative to one another about a hub, and the hub may have a cavity formed therein that is sized and shaped to receive the shaft. A spring may be positioned in the hub to selectively retain the shaft therein.

According to another aspect, a method of surgically preparing a patient’s femur and tibia during an orthopaedic surgical knee procedure includes resecting a distal end of the patient’s femur and a proximal end of the patient’s tibia and thereafter inserting a spacer block of a boundary assessment instrument between the resected distal end of the patient’s femur and the resected proximal end of the patient’s tibia. A tibial alignment rod of the boundary assessment instrument is aligned with an ankle of the patient. With the tibial alignment rod of the boundary assessment instrument aligned with the patient’s ankle, a varus/valgus angle is determined from a varus/valgus angle gauge of the boundary assessment instrument. A femoral alignment rod of the boundary assessment instrument is aligned with a hip of the patient. With the femoral alignment rod of the boundary assessment instrument aligned with the patient’s hip, a hip-knee-ankle angle is determined from a hip-knee-ankle angle gauge of the boundary assessment instrument.

In an example, the patient’s femur and tibia is positioned in extension prior to insertion of the spacer block.

In an example, the tibial alignment rod is aligned with the patient’s ankle during alignment of the femoral alignment rod with the patient’s hip.

In another example, the tibial alignment rod is rotated relative to the spacer block to align the tibial alignment rod with the patient’s ankle, and the femoral alignment rod is rotated relative to the spacer block to align the femoral alignment rod with the patient’s hip.

The femoral alignment rod may be rotated relative to both the spacer block and the tibial alignment rod to align the femoral alignment rod with the patient’s hip.

In an example, the boundary assessment instrument includes a varus/valgus pointer, and the varus/valgus angle gauge is moved relative to the varus/valgus pointer.

The varus/valgus angle gauge may include a plurality of angle indicators each of which corresponds to a different varus/valgus angle, and the varus/valgus angle gauge may be moved relative to the varus/valgus pointer such that the varus/valgus pointer aligns with one of the plurality of angle indicators.

In an example, the varus/valgus angle gauge is secured to the tibial alignment rod and is moved during movement of the tibial alignment rod.

In an example, the boundary assessment instrument includes a hip-knee-ankle pointer. Aligning the tibial alignment rod may include rotating one of the hip-knee-ankle angle gauge and the hip-knee-ankle pointer with the tibial alignment rod, and aligning the femoral alignment rod may include moving the other of the hip-knee-ankle angle gauge and the hip-knee-ankle pointer.

The hip-knee-ankle angle gauge may include a plurality of angle indicators each of which corresponds to a different hip-knee-ankle angle, and aligning the femoral alignment rod may include moving one of the hip-knee-ankle angle gauge and the hip-knee-ankle pointer relative to the other of the hip-knee-ankle angle gauge and the hip-knee-ankle pointer such that the hip-knee-ankle pointer aligns with one of the plurality of angle indicators.

The boundary assessment instrument may include a hip-knee-ankle pointer rotatably secured to the shaft, with the hip-knee-ankle angle gauge being secured to the femoral alignment rod. The hip-knee-ankle angle gauge may be moved relative to the hip-knee-ankle pointer during movement of the femoral alignment rod.

According to another aspect, an orthopaedic surgical instrument assembly for use during an orthopaedic knee replacement procedure includes a varus/valgus pointer extending inferiorly, a femoral alignment arm, and a tibial alignment arm. The femoral alignment arm extends superiorly. The orthopaedic surgical instrument also has a hip-knee-ankle pointer and a hip-knee-ankle arm segment extending superiorly, the hip-knee-ankle arm segment having a hip-knee-ankle angle gauge. The tibial alignment arm is rotatably coupled to the varus/valgus pointer and includes a varus/valgus arm segment extending inferiorly, the varus/valgus arm segment having a varus/valgus angle gauge. Rotation of the tibial alignment arm causes the varus/valgus angle gauge to be moved relative to the varus/valgus pointer.

According to another aspect, a method of surgically preparing a patient’s femur and tibia during an orthopaedic surgical knee procedure includes inserting a spacer block of a boundary assessment instrument between an unresected distal end of the patient’s femur and an unresected proximal end of the patient’s tibia. A tibial alignment rod of the boundary assessment instrument is aligned with an ankle of the patient. With the tibial alignment rod of the boundary assessment instrument aligned with the patient’s ankle, a varus/valgus angle is determined from a varus/valgus angle gauge of the boundary assessment instrument. A femoral alignment rod of the boundary assessment instrument is aligned with a hip of the patient. With the femoral alignment rod of the boundary assessment instrument aligned with the patient’s hip, a hip-knee-ankle angle is determined from a hip-knee-ankle angle gauge of the boundary assessment instrument.

In an example, the patient’s femur and tibia is positioned in extension prior to insertion of the spacer block.

In an example, the tibial alignment rod is aligned with the patient’s ankle during alignment of the femoral alignment rod with the patient’s hip.

In another example, the tibial alignment rod is rotated relative to the spacer block to align the tibial alignment rod with the patient’s ankle, and the femoral alignment rod is rotated relative to the spacer block to align the femoral alignment rod with the patient’s hip.

The femoral alignment rod may be rotated relative to both the spacer block and the tibial alignment rod to align the femoral alignment rod with the patient’s hip.

In an example, the boundary assessment instrument includes a varus/valgus pointer secured to the spacer block, and the varus/valgus gauge is moved relative to the varus/valgus pointer.

The varus/valgus angle gauge may include a plurality of angle indicators each of which corresponds to a different varus/valgus angle, and the varus/valgus angle gauge is moved relative to the varus/valgus pointer such that the varus/valgus pointer aligns with one of the plurality of angle indicators.

In an example, the varus/valgus angle gauge is secured to the tibial alignment rod and is moved during movement of the tibial alignment rod.

In an example, the boundary assessment instrument includes a hip-knee-ankle pointer, and the hip-knee-ankle angle gauge is moved relative to the hip-knee-ankle pointer.

The hip-knee-ankle angle gauge may include a plurality of angle indicators each of which corresponds to a different hip-knee-ankle angle, and the hip-knee-ankle pointer is moved relative to the hip-knee-ankle angle gauge such that the hip-knee-ankle pointer aligns with one of the plurality of angle indicators.

The boundary assessment instrument may include a hip-knee-ankle pointer, with the hip-knee-ankle angle gauge being secured to the femoral alignment rod. The hip-knee-ankle angle gauge is moved relative to the hip-knee-ankle pointer during movement of the femoral alignment rod.

According to another aspect, an orthopaedic surgical instrument assembly for use during an orthopaedic knee replacement procedure includes a spacer block configured to be positioned between a resected femur and a resected tibia of a patient and a shaft extending anteriorly away from the spacer block. The spacer block has a flat superior surface and a flat inferior surface. A varus/valgus pointer extends inferiorly away from the shaft. A femoral alignment arm is rotatably coupled to the shaft. The femoral alignment arm extends superiorly away from the shaft. A hip-knee-ankle pointer is located superior to the shaft. A hip-knee-ankle arm segment is located superior to the shaft and has a hip-knee-ankle angle gauge. A tibial alignment arm is rotatably coupled to the shaft and has a varus/valgus arm segment extending inferiorly away from the shaft. The varus/valgus arm segment has a varus/valgus angle gauge. Rotation of the tibial alignment arm causes the varus/valgus angle gauge to be moved relative to the varus/valgus pointer.

In an example, a femoral alignment rod extends superiorly away from a superior end of the femoral alignment arm, and a tibial alignment rod extends inferiorly away from an inferior end of the tibial alignment arm.

In another example, the femoral alignment rod is extendable and retractable relative to the superior end of the femoral alignment arm, and the tibial alignment rod is extendable and retractable relative to the interior end of the tibial alignment arm.

In another example, a longitudinal axis of the varus/valgus pointer is orthogonal to the flat superior surface of the spacer block.

In an example, the flat superior surface of the spacer block is parallel to the flat inferior surface of the spacer block.

The varus/valgus angle gauge may include a plurality of angle indicators each of which corresponds to a different varus/valgus angle. Rotation of the tibial alignment arm causes the varus/valgus angle gauge to be moved relative to the varus/valgus pointer such that the varus/valgus pointer aligns with one of the plurality of angle indicators.

The femoral alignment arm and the tibial alignment arm may form an anteroposterior angle, and the anteroposterior angle may be between 155 degrees to 175 degrees.

In an example, the hip-knee-ankle arm segment is integrally formed with the femoral alignment arm such that the hip-knee-ankle arm segment rotates with the femoral alignment arm.

In another example, the varus/valgus pointer includes a locking knob configured to lock the varus/valgus pointer to the varus/valgus arm segment such that rotation of the tibial alignment arm relative to the varus/valgus pointer is blocked.

### BRIEF DESCRIPTION OF THE DRAWINGS

The detailed description particularly refers to the following figures, in which

FIG. 1 is a fragmentary perspective view of a boundary assessment instrument for use in the surgical preparation of a patient’s femur and tibia during performance of an orthopaedic knee procedure;

FIG. 2 is a fragmentary front view of the boundary assessment instrument, note the flexion spacer block has been removed in FIG. 2 for clarity of description;

FIG. 3 is a fragmentary side view of the boundary assessment instrument, note the flexion spacer block has been removed in FIG. 3 for clarity of description;

FIG. 4 is a front view of the boundary assessment instrument of FIG. 1 installed in the patient’s knee, note the flexion spacer block has been removed in FIG. 4 for clarity of description;

FIG. 5 is an enlarged fragmentary view similar to FIG. 4, note the flexion spacer block has been removed in FIG. 5 for clarity of description;

FIG. 6 is a front view of a resected femur and tibia that illustrates a manner for determining the varus/valgus angle of a patient’s tibia;

FIG. 7 is a perspective view of another boundary assessment instrument for use in the surgical preparation of a patient’s femur and tibia during performance of an orthopaedic knee procedure;

FIG. 8 is a perspective view of the boundary assessment instrument of FIG. 7 with the spacer blocks removed for clarity of description;

FIG. 9 is a side view of the boundary assessment instrument of FIG. 8;

FIG. 10 is an enlarged fragmentary front view of the boundary assessment instrument of FIG. 8; and

FIG. 11 is an enlarged fragmentary side view of the boundary assessment instrument of FIG. 8.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, proximal, distal, etcetera, may be used throughout the specification in reference to the orthopaedic implants and surgical instruments described herein as well as in reference to the patient’s natural anatomy. Such terms have well-understood meanings in both the study of anatomy and the field of orthopaedics. Use of such anatomical reference terms in the written description and claims is intended to be consistent with their well-understood meanings unless noted otherwise.

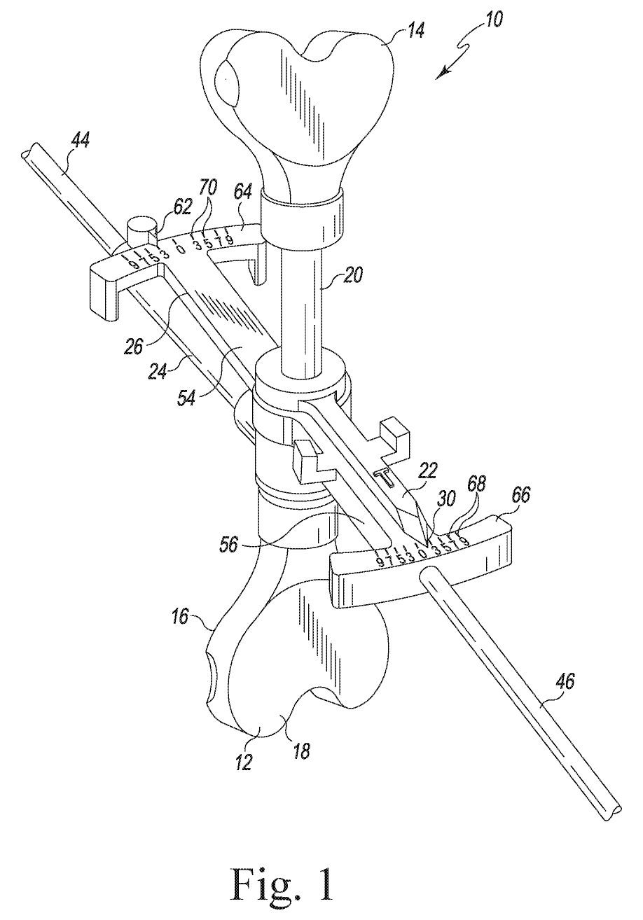

Referring to FIGS. 1-3, an orthopaedic surgical instrument 10 - in the form of a boundary assessment instrument - for use in the surgical preparation of a patient’s femur and tibia during performance of an orthopaedic knee procedure is shown. As will be described below in more detail, the boundary assessment instrument 10 may be used by a surgeon to confirm the alignment of the patient’s femur and tibia are within preoperatively-determined parameters. For instance, surgeons generally set upper boundaries or “maximums” in regard to the overall hip-knee-ankle angle of the patient’s leg or the varus/valgus angle of the patient’s tibia. For example, the varus/valgus angle of the patient’s tibia is generally limited to 7 degrees or less. Similarly, the overall hip-knee-ankle angle of the patient’s leg is likewise generally limited to 7 degrees or less. The boundary assessment instrument 10 may be used by a surgeon to efficiently and accurately determine if such limits are being intraoperatively achieved.

The boundary assessment instrument 10 includes an extension spacer block 12 and a flexion spacer block 14. The spacer blocks 12, 14 may be inserted into the joint gap after performance of a distal cut on the patient’s femur 120 and a proximal cut on the patient’s tibia 130 to assess the extension gap and the flexion gap, respectively. In some embodiments, the instrument 10 may be used with a resected tibia and a native femur. Each of the spacer blocks 12, 14 has a flat superior surface 16 and an opposite, parallel flat inferior surface 18 for conformance to the planar resected surfaces of the femur 120 and tibia 130. It should be appreciated that one or both of the spacer blocks 12, 14 may be fitted with shims to alter the thickness of the block. As will become apparent below, the boundary assessment functions of the instrument 10 are performed with the patient’s knee positioned in extension. As such, the features of the instrument 10 utilized during boundary assessment are presented to the surgeon in relation to use of the extension spacer block 12.

As can be seen in FIG. 1, a shaft 20 extends anteriorly away from the extension spacer block 12. The shaft 20 supports the various components of the boundary assessment instrument 10. Specifically, a varus/valgus pointer 22, a femoral alignment arm 24, and a tibial alignment arm 26 are all secured to the shaft 20. As will be discussed below in more detail, the femoral alignment arm 24 and the tibial alignment arm 26 are rotatable about the shaft 20 so that a femoral alignment rod 44 and a tibial alignment rod 46 may be aligned with the patient’s hip 124 and ankle 134, respectively.

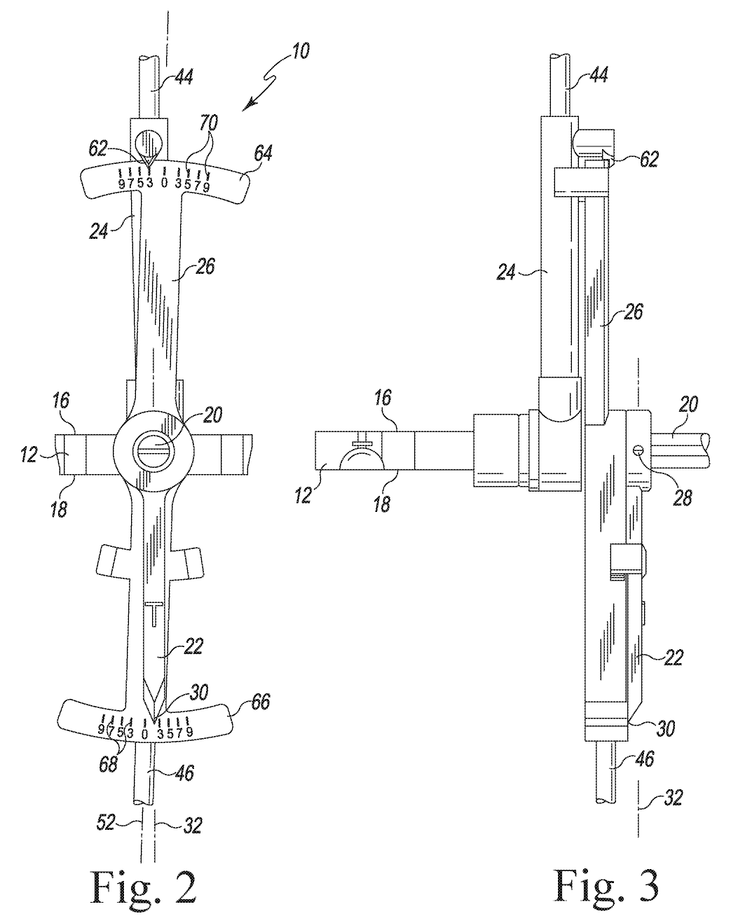

The varus/valgus pointer 22 is non-rotatably secured to the shaft 20 and is thus fixed in position on the shaft 20. As can be seen in FIG. 3, the superior end of the varus/valgus pointer 22 is pinned to the shaft 20 via a locking pin 28. As can be seen best in FIGS. 2 and 3, the varus/valgus pointer 22 extends inferiorly away from the shaft 20 and terminates at a pointed tip 30. As can also be seen best in FIGS. 2 and 3, the longitudinal axis 32 of the varus/valgus pointer 22 is orthogonal to the flat superior surface 16 and the flat inferior surface 18 of the spacer block 12.

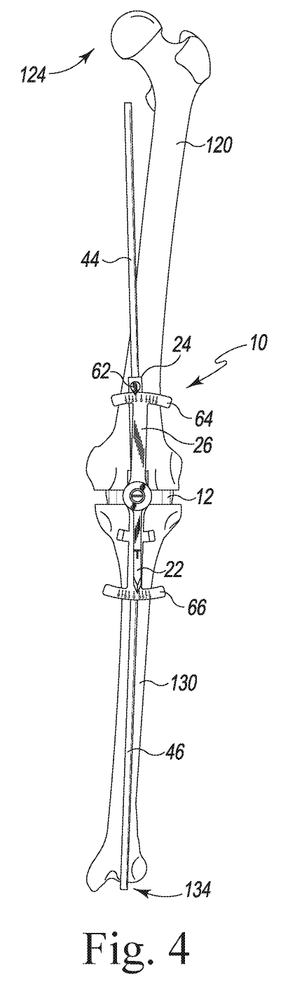

As alluded to above, the tibial alignment arm 26 has a tibial alignment rod 46 secured to its inferior end. As can be seen in FIG. 4, the tibial alignment rod 46 extends inferiorly away from the tibial alignment arm 26 toward the patient’s ankle 134. The tibial alignment arm 26 may be rotated about the shaft 20 to align the alignment rod 46 in a position relative to the patient’s ankle 134 so that the alignment of the patient’s femur 120 and tibia 130 may be assessed. Specifically, the tibial alignment arm 26 may be rotated relative to the shaft 20 to position the tibial alignment rod 46 along the mechanical axis of the patient’s tibia 130 such that it is aligned with the patient’s ankle 134, while the varus/valgus pointer 22 extends orthogonally relative to the planar resected surface 136 of the tibia 130 because of the fixed orthogonal position of the varus/valgus pointer 22 relative to the flat inferior surface 18 of the extension spacer block 12. In that position, the boundary assessment instrument 10 allows the surgeon to determine the varus/valgus angle of the installed extension spacer block 12 (and hence the prosthetic implant that would be implanted in the block’s place). In particular, as shown graphically in FIG. 6, the varus/valgus angle 150 is defined between the anatomic axis 152 of the tibia 130 and an axis 154 perpendicular to the planar resected surface 136 of the tibia 130. By virtue of being fixed orthogonally to the flat inferior surface 18 of the spacer block 12, the varus/valgus pointer 22 is aligned with the axis 154.

Positioning the tibial alignment rod 46 along the patient’s tibia 130 in alignment with the patient’s ankle 134 allows the boundary assessment instrument 10 to identify the anatomic axis 152 of the tibia 130 since the two axes coincide with one another in that position. Thus, when the spacer block 12 is installed and the tibial alignment rod 46 is aligned with the patient’s ankle 134, the varus/valgus angle of the patient’s tibia 130 may be determined by measuring the included angle between the longitudinal axis 32 of the varus/valgus pointer 22 and the longitudinal axis 52 of the tibial alignment rod 46. The varus/valgus angle of the patient’s tibia 130 informs the varus/valgus positioning of the patient’s knee.

As also alluded to above, the femoral alignment arm 24 has a femoral alignment rod 44 secured to its superior end. As can be seen in FIG. 4, the femoral alignment rod 44 extends superiorly away from the femoral alignment arm 24 toward the patient’s hip 124. The femoral alignment arm 24 may be rotated about the shaft 20 to align the alignment rod 44 in a position relative to the patient’s hip 124 so that the alignment of the patient’s femur 120 and tibia 130 may be assessed. Specifically, the femoral alignment arm 24 may be rotated relative to the shaft 20 to position the femoral alignment rod 44 along an axis that it is aligned with the patient’s hip 124 (more specifically, the centre of the femoral head of the patient’s hip 124). As will be described below, doing so allows the surgeon to determine the overall hip-knee-ankle angle of the patient’s leg created by the installed extension spacer block 12 (and hence the prosthetic implant that would be implanted in the block’s place).

As can be seen in FIGS. 1-3, the tibial alignment arm 26 has a hip-knee-ankle arm segment 54 extending superiorly away from the shaft 20 and a varus/valgus arm segment 56 extending inferiorly away from the shaft 20. The two arm segments 54, 56 are linearly aligned with both one another and the tibial alignment rod 46. In particular, the longitudinal axes of both arm segments 54, 56 are colinear with one another and the tibial alignment rod 46 and thus lie on the same line as the alignment rod’s longitudinal axis 52 (see FIG. 2).

As can be seen in FIGS. 1 and 2, the varus/valgus arm segment 56 of the tibial alignment arm 26 has a varus/valgus angle gauge 66 secured to its inferior end. In the example described herein, the varus/valgus angle gauge 66 is integrally formed in the tibial alignment arm 26. The varus/valgus angle gauge 66 moves radially relative to the varus/valgus pointer 22 during rotation of the tibial alignment arm 26 about the shaft 20. The varus/valgus angle gauge 66 has indicia in the form of a plurality of angle indicators 68 radially engraved, printed, or otherwise formed thereon. Each of the plurality of angle indicators 68 represents a varus/valgus angle (e.g., left or right – 0 degrees to 9 degrees) for presentation to a surgeon during use of the boundary assessment instrument 10. In particular, and as will be described in more detail below, the surgeon may read the output from the varus/valgus angle gauge 66 to determine the varus/valgus angle of the spacer block 12 installed in the extension gap (i.e., between the resected distal femur 120 and resected proximal tibia 130).

As can also be seen in FIGS. 1 and 2, the hip-knee-ankle arm segment 54 of the tibial alignment arm 26 has a hip-knee-ankle angle gauge 64 secured to its superior end. In the example described herein, the hip-knee-ankle angle gauge 64 is integrally formed in the tibial alignment arm 26. The hip-knee-ankle angle gauge 64 moves radially relative to a hip-knee-ankle pointer 62 secured to the femoral alignment arm 24 during rotation of the tibial alignment arm 26 about the shaft 20. In addition, the hip-knee-ankle pointer 62 moves radially relative to the hip-knee-ankle angle gauge 64 during rotation of the femoral alignment arm 24 about the shaft 20. The hip-knee-ankle angle gauge 64 has indicia in the form of a plurality of angle indicators 70 radially engraved, printed, or otherwise formed thereon. Each of the plurality of angle indicators 70 represents a hip-knee-ankle angle (e.g., left or right – 0 degrees to 9 degrees) for presentation to a surgeon during use of the boundary assessment instrument 10. In particular, and as will be described in more detail below, the surgeon may read the output from the hip-knee-ankle angle gauge 64 to determine the overall hip-knee-ankle angle of the patient’s leg created by the spacer block 12 installed in the extension gap (i.e., between the resected distal femur 120 and resected proximal tibia 130).

The boundary assessment instrument 10 may be used both by surgeons with a preference for mechanical alignment techniques and surgeons with a preference for kinematic alignment techniques. Broadly, mechanical alignment techniques involve determining the resection planes based on a pre-determined angle as a function of mechanical alignment, whereas kinematic alignment techniques involve determining the resection planes as a function of the native, pre-disease state of the patient’s knee. In either case, the surgeon sets a preoperative boundary or “maximum” allowable varus/valgus angle and a preoperative boundary or “maximum” allowable hip-knee-ankle angle. The boundary assessment instrument 10 may be used by the surgeon to intraoperatively confirm both angles are within the boundaries set by the surgeon.

In operation, the surgeon may utilize the boundary assessment instrument 10 during performance of an orthopaedic knee procedure to prepare the distal end 122 of the patient’s femur 120 and the proximal end 132 of the patient’s tibia 130 to receive prosthetic femoral and tibial components. In doing so, the surgeon may utilize the boundary assessment instrument 10 to intraoperatively assess resections to the patient’s femur 120 and tibia 130 to ensure a desired placement of the prosthetic femoral and tibial components.

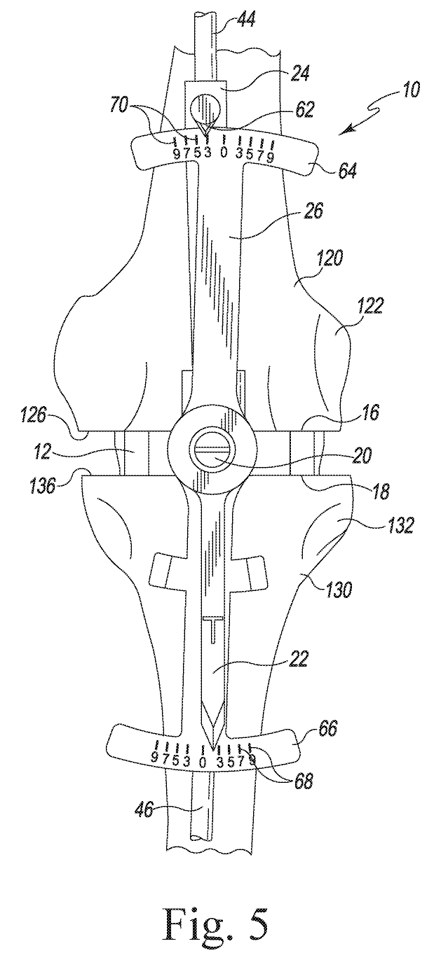

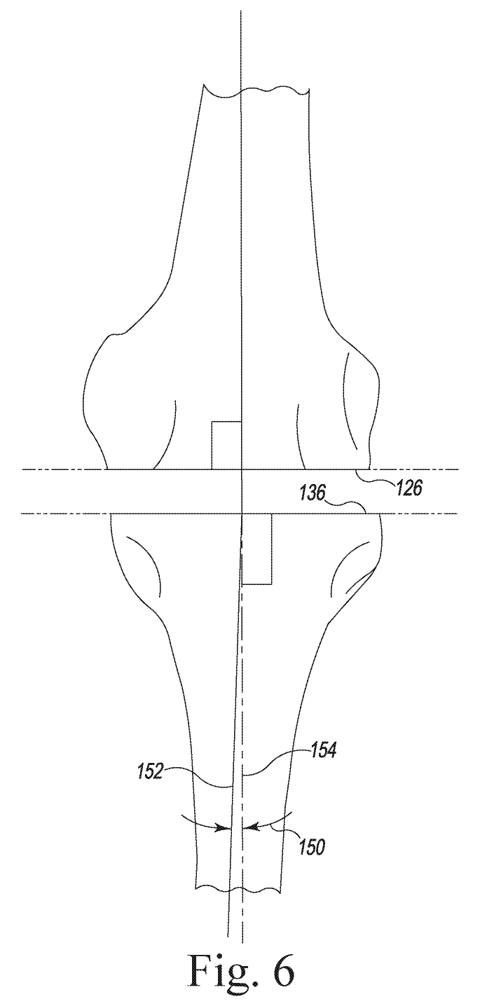

During such an orthopaedic surgical procedure, the surgeon performs a distal femoral resection on the patient’s femur 120 thereby producing a planar resected surface 126 on the distal end 122 of the patient’s femur 120, as shown in FIGS. 4 and 5. As also shown in FIGS. 4 and 5, prior to, or subsequent to, performance of the distal femoral resection, the surgeon also performs a tibial resection on the patient’s tibia 130 thereby producing a planar resected surface 136 on the proximal end 132 of the patient’s tibia 130.

Subsequent to performing both resections, the surgeon orientates the patient’s femur 120 and tibia 130 such that the patient’s knee is positioned in extension, as shown in FIGS. 4 and 5. With the patient’s knee positioned in extension, the surgeon installs the boundary assessment instrument 10 to intraoperatively assess the resections to the patient’s femur 120 and tibia 130. To do so, the spacer block 12 is inserted into the joint gap created by the distal cut on the patient’s femur 120 and the proximal cut on the patient’s tibia 130. During such installation, the flat superior surface 16 of the spacer blocks 12 contacts with the planar resected surface 126 of the distal end 122 of the patient’s femur 120 and the opposite, parallel flat inferior surface 18 of the extension spacer block 12 contacts the planar resected surface 136 of the proximal end 132 of the patient’s tibia 130.

The surgeon may then use the installed boundary assessment instrument 10 to confirm the alignment of the patient’s femur 120 and tibia 130 created by the resections is within preoperatively-determined upper boundaries or “maximums” in regard to the overall hip-knee-ankle angle of the patient’s leg and the varus/valgus angle of the installed extension spacer block 12 (i.e., the varus/valgus angle of the patient’s tibia 130). To do so, the surgeon rotates the tibial alignment arm 26 relative to the shaft 20 and positions the tibial alignment rod 46 along the mechanical axis of the patient’s tibia 130 such that it is aligned with the patient’s ankle 134, as shown in FIG. 4. Doing so moves the varus/valgus angle gauge 66 radially relative to the stationary varus/valgus pointer 22 such that the varus/valgus pointer 22 aligns with one of the gauge’s angle indicators 68 that is indicative of the varus/valgus angle (e.g., left or right – 0 degrees to 9 degrees). Once so aligned, the surgeon reads the output from the varus/valgus angle gauge 66 to determine the varus/valgus angle of the spacer block 12 installed in the extension gap (and therefore the tibial resection).

With the tibial alignment rod 46 aligned with the patient’s ankle 134, the surgeon then rotates the femoral alignment arm 24 relative to the shaft 20 and positions the femoral alignment rod 44 such that it is aligned with the patient’s hip 124, as shown in FIG. 4. Doing so moves the hip-knee-ankle pointer 62 radially relative to the stationary hip-knee-ankle angle gauge 64 such that the hip-knee-ankle pointer 62 aligns with one of the gauge’s angle indicators 70 that is indicative of the overall hip-knee-ankle angle of the patient’s leg created by the spacer block 12 installed in the extension gap. Because the hip-knee-ankle angle gauge 64 is fixed to the tibial alignment arm 26 to rotate therewith, the hip-knee-ankle angle gauge 64 is aligned with the mechanical axis of the patient’s tibia 130. Once so aligned, the surgeon reads the output from the hip-knee-ankle angle gauge 64 to determine the overall hip-knee-ankle angle of the patient’s leg created by the spacer block 12 installed in the extension gap.

Once the surgeon has determined the varus/valgus angle and the overall hip-knee-ankle angle of the patient’s leg created by the spacer block 12 installed in the extension gap, the surgeon can compare the same to preoperatively-determined boundaries for both measurements. For example, the varus/valgus angle of the patient’s tibia is generally limited to 7 degrees or less. Similarly, the overall hip-knee-ankle angle is likewise generally limited to 7 degrees or less. If necessary, the surgeon can perform recuts to adjust the angle of either the planar resected surface 126 of the patient’s femur 120 or the planar resected surface 136 of the patient’s tibia 130, or both. The surgeon may then re-evaluate the alignment created by the recuts by reinstalling the boundary assessment instrument 10 and measuring the revised varus/valgus angle and hip-knee-ankle angle.

Once the surgeon is satisfied with the extension gap assessment, the surgeon may then perform the remaining surgical steps to complete the orthopaedic knee procedure.

It should be appreciated that although the boundary assessment instrument 10 is herein described as being used after both the planar resected surface 126 of the patient’s femur 120 and the planar resected surface 136 of the patient’s tibia 130 have been formed, the instrument 10 may also be used prior to resection of the patient’s femur 120 and tibia 130. For example, the boundary assessment instrument 10 may be inserted on top of the resection planes of tibial resection block and the femoral resection block to verify the alignment of the patient’s leg prior to resection of the patient’s femur 120 and tibia 130 and then subsequently used to adjust the angle between the two resection planes to a defined angle pre-resection.

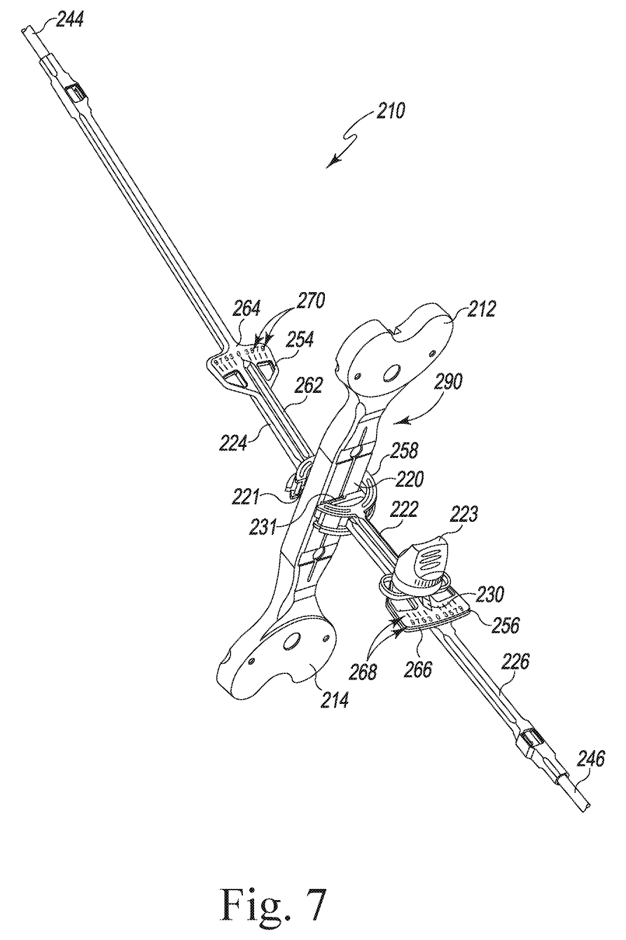

Referring now to FIGS. 7-11 and as discussed further below, in another embodiment, an orthopaedic surgical instrument 210 - in the form of a boundary assessment instrument - for use in the surgical preparation of a patient’s femur and tibia during performance of an orthopaedic knee procedure is shown. The boundary assessment instrument 210 of FIGS. 7-11 is substantially similar to the boundary assessment instrument 10 shown in FIGS. 1-5 and described herein. Accordingly, similar reference numbers are used in the description of the boundary assessment instrument 210 to indicate features that are common between the boundary assessment instrument 10 and the boundary assessment instrument 210. The description of the boundary assessment instrument 10 is incorporated by reference to apply to the boundary assessment instrument 210, except in instances when it conflicts with the specific description and the drawings of the boundary assessment instrument 210.

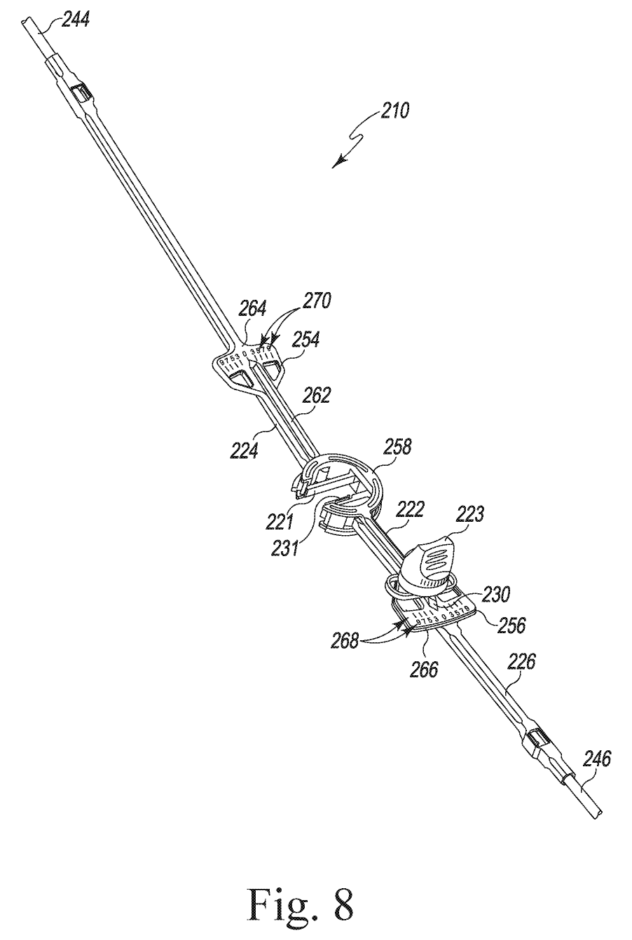

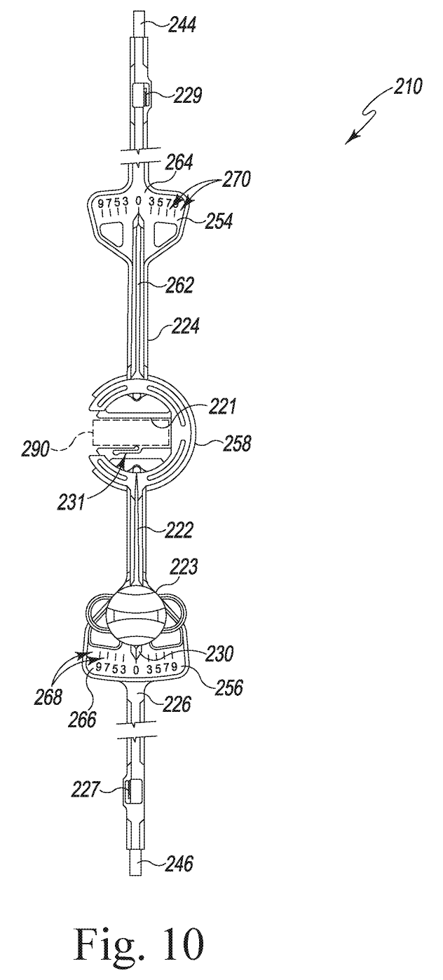

The boundary assessment instrument 210 includes a base illustratively in the form of a removable spacer instrument 290, as shown in FIG. 7. The removable spacer instrument 290 includes an extension spacer block 212, a flexion spacer block 214, and a shaft 220, as shown in FIG. 7. As compared to the boundary assessment instrument 10, the extension spacer block 212, the flexion spacer block 214, and the shaft 220 are removable from the boundary assessment instrument 210. It should be appreciated that the spacer blocks 212, 214 and the shaft 220 are removed from FIGS. 8-11 for clarity of description. The spacer blocks 212, 214 and the shaft 220 are removably coupled to a hub 258 of the boundary assessment instrument 210, as shown in FIG. 10. For example, the shaft 220 can be inserted into a cavity 221 formed in the hub 258. The boundary assessment instrument 210 includes a spring 231 located in the cavity 221 to maintain the position of the removable spacer instrument 290 relative to the hub 258 while the shaft 220 is positioned in the cavity 221, as shown in FIGS. 7 and 10.

As shown in FIG. 8, the boundary assessment instrument 210 includes a varus/valgus pointer 222, a femoral alignment arm 224, and a tibial alignment arm 226. As will be discussed below in more detail, the femoral alignment arm 224 and the tibial alignment arm 226 are rotatable relative to one another so that a femoral alignment rod 244 and a tibial alignment rod 246 may be aligned with the patient’s hip 124 and ankle 134, respectively.

The varus/valgus pointer 222 is fixed to the hub 258. As can be seen best in FIGS. 7 and 10, the varus/valgus pointer 222 extends inferiorly away from the shaft 220 and the hub 258 and terminates at a pointed tip 230.

As alluded to above, the tibial alignment arm 226 has the tibial alignment rod 246 secured to its inferior end. As can be seen in FIG. 8, the tibial alignment rod 246 extends inferiorly away from the tibial alignment arm 226 toward the patient’s ankle 134. The tibial alignment arm 226 may be rotated relative to (i.e., independent of) the femoral alignment arm 224 to align the tibial alignment rod 246 in a position relative to the patient’s ankle 134 so that the alignment of the patient’s femur 120 and tibia 130 may be assessed. Specifically, the tibial alignment arm 226 may be rotated to position the tibial alignment rod 246 along the mechanical axis of the patient’s tibia 130 such that it is aligned with the patient’s ankle 134, while the varus/valgus pointer 222 extends orthogonally relative to the planar resected surface 136 of the tibia 130 because of the fixed orthogonal position of the varus/valgus pointer 222 relative to the flat inferior surface of the extension spacer block 12.

As also alluded to above, the femoral alignment arm 224 has the femoral alignment rod 244 secured to its superior end. As can be seen in FIG. 8, the femoral alignment rod 244 extends superiorly away from the femoral alignment arm 224 toward the patient’s hip 124. The femoral alignment arm 224 may be rotated relative to the tibial alignment arm 226 to align the femoral alignment rod 244 in a position relative to the patient’s hip 124 so that the alignment of the patient’s femur 120 and tibia 130 may be assessed. Specifically, the femoral alignment arm 224 may be rotated to position the femoral alignment rod 244 along an axis that is aligned with the patient’s hip 124. As will be described below, doing so allows the surgeon to determine the overall hip-knee-ankle angle of the patient’s leg created by the installed extension spacer block 212 (and hence the prosthetic implant that would be implanted in the block’s place).

As can be seen in FIGS. 7 and 10, the femoral alignment arm 224 has a hip-knee-ankle arm segment 254 extending superiorly away from the shaft 220 and the hub 258. The tibial alignment arm 226 has a varus/valgus arm segment 256 extending inferiorly away from the shaft 220 and the hub 258. The hip-knee-ankle arm segment 254 is linearly aligned with the femoral alignment rod 244, and the varus/valgus arm segment 256 is linearly aligned with the tibial alignment rod 246. In particular, the longitudinal axis of the hip-knee-ankle arm segment 254 is colinear with the femoral alignment rod 244, and the longitudinal axis of the varus/valgus arm segment 256 is colinear with the tibial alignment rod 246 and thus lies on the same line as the alignment rod’s longitudinal axis 52 (see FIG. 2).

As can be seen in FIGS. 8 and 10, the varus/valgus arm segment 256 of the tibial alignment arm 226 has a varus/valgus angle gauge 266. In the example described herein, the varus/valgus angle gauge 266 is integrally formed in the tibial alignment arm 226. The varus/valgus angle gauge 266 moves radially relative to the varus/valgus pointer 222 during rotation of the tibial alignment arm 226. The varus/valgus angle gauge 266 has indicia in the form of a plurality of angle indicators 268 radially engraved, printed, or otherwise formed thereon. Each of the plurality of angle indicators 268 represents a varus/valgus angle (e.g., left or right – 0 degrees to 9 degrees) for presentation to a surgeon during use of the boundary assessment instrument 210. In particular, the surgeon may read the output from the varus/valgus angle gauge 266 to determine the varus/valgus angle of the spacer block 212 installed in the extension gap (i.e., the varus/valgus angle of the patient’s tibia 130).

As shown in FIGS. 8 and 10, the varus/valgus arm segment 256 includes a locking knob 223. The locking knob 223 is coupled to the varus/valgus arm segment 256 to move therewith. The locking knob 223 may be rotated by the surgeon to lock and unlock the position of the varus/valgus pointer 222 relative to the varus/valgus arm segment 256 of the tibial alignment arm 226. In this way, relative movement between the varus/valgus pointer 222 and the varus/valgus arm segment 256 of the tibial alignment arm 226 is blocked or freely allowed.

As can also be seen in FIGS. 8 and 10, the hip-knee-ankle arm segment 254 of the femoral alignment arm 224 has a hip-knee-ankle angle gauge 264. In the example described herein, the hip-knee-ankle angle gauge 264 is integrally formed in the femoral alignment arm 224. The hip-knee-ankle angle gauge 264 moves radially relative to a hip-knee-ankle pointer 262 coupled to the hub 258 during rotation of the femoral alignment arm 224. The hip-knee-ankle angle gauge 264 has indicia in the form of a plurality of angle indicators 270 radially engraved, printed, or otherwise formed thereon. Each of the plurality of angle indicators 270 represents a hip-knee-ankle angle (e.g., left or right – 0 degrees to 9 degrees) for presentation to a surgeon during use of the boundary assessment instrument 210. In particular, the surgeon may read the output from the hip-knee-ankle angle gauge 264 to determine the overall hip-knee-ankle angle of the patient’s leg created by the spacer block 212 installed in the extension gap (i.e., between the resected distal femur 120 and resected proximal tibia 130).

The hip-knee-ankle pointer 262 is coupled to the tibial alignment arm 226 to rotate with the tibial alignment arm 226. In this way, the hip-knee-ankle pointer 262 rotates with the tibial alignment arm 226 relative to the femoral alignment arm 224. Because the hip-knee-ankle pointer 262 rotates with the tibial alignment arm 226, the hip-knee-ankle pointer 262 may be positioned along the mechanical axis of the patient’s tibia 130.

As shown in FIGS. 8 and 10, the tibial alignment rod 246 is extendable and retractable relative to the tibial alignment arm 226. For example, the tibial alignment rod 246 may be grasped by the surgeon and pulled inferiorly from the tibial alignment arm 226 thereby extending the tibial alignment rod 246. In this way, the combined length of the tibial alignment arm 226 and the tibial alignment rod 246 is increased. As another example, the tibial alignment rod 246 may be grasped by the surgeon and pushed superiorly into the tibial alignment arm 226 thereby retracting the tibial alignment rod 246. In this way, the combined length of the tibial alignment arm 226 and the tibial alignment rod 246 is decreased.

The tibial alignment arm 226 includes a spring 227, as shown in FIG. 10. The spring 227 acts against the tibial alignment rod 246 to maintain a position of the tibial alignment rod 246 relative to the tibial alignment arm 226 until acted upon by the surgeon.

As shown in FIGS. 8 and 10, the femoral alignment rod 244 is extendable and retractable relative to the femoral alignment arm 224. For example, the femoral alignment rod 244 may be grasped by the surgeon and pulled superiorly from the femoral alignment arm 224 thereby extending the femoral alignment rod 244. In this way, the combined length of the femoral alignment arm 224 and the femoral alignment rod 244 is increased. As another example, the femoral alignment rod 244 may be grasped by the surgeon and pushed inferiorly into the femoral alignment arm 224 thereby retracting the femoral alignment rod 244. In this way, the combined length of the femoral alignment arm 224 and the femoral alignment rod 244 is decreased.

The femoral alignment arm 224 includes a spring 229, as shown in FIG. 10. The spring 229 acts against the femoral alignment rod 244 to maintain a position of the femoral alignment rod 244 relative to the femoral alignment arm 224 until acted upon by the surgeon.

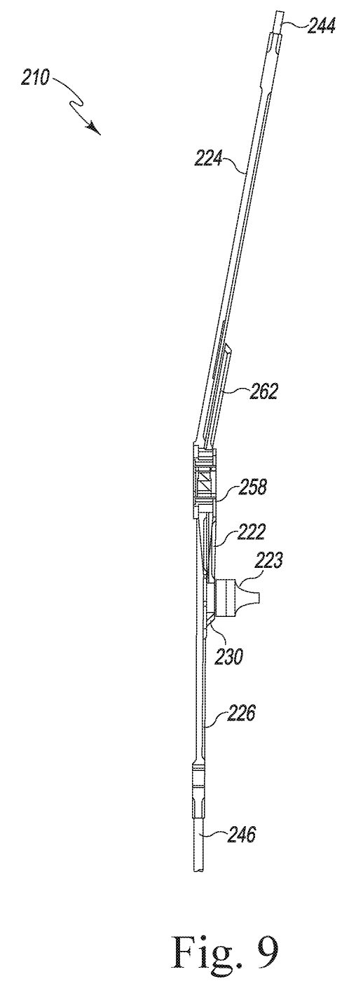

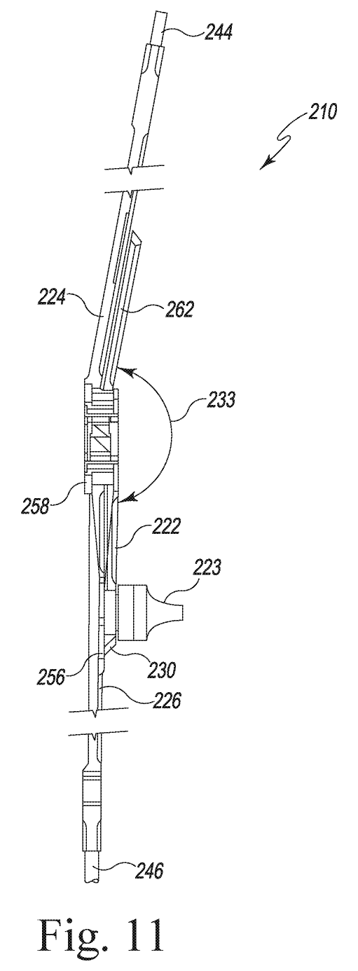

As shown in FIGS. 9 and 11, the femoral alignment arm 224 and the tibial alignment arm 226 form an anteroposterior angle 233. The tibial alignment arm 226 (and the varus/valgus pointer 222) extends inferiorly away from the shaft 220, and the femoral alignment arm 224 (and the hip-knee-ankle pointer 262) extends superiorly and anteriorly away from the shaft 220. In this way, the tibial alignment arm 226 and the femoral alignment arm 224 are not colinear with one another. The anteroposterior angle 233 is between about 155 degrees to about 180 degrees. In some embodiments, the anteroposterior angle 233 is between about 155 degrees to about 175 degrees. The anteroposterior angle 233 allows the femoral alignment arm 224 to clear the patient’s thigh during use of the boundary assessment instrument 210.

In some embodiments, the femoral alignment arm 224 can be disconnected from the hub 258 so that the boundary assessment instrument 210 can be used on the patient’s tibia 130 alone.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. An orthopaedic surgical instrument assembly for use during an orthopaedic knee replacement procedure, comprising:
an elongated base having a posterior end configured to be positioned between a femur and a tibia of a patient and a shaft extending anteriorly away from the posterior end,
a femoral alignment tool extending superiorly away from the shaft, the femoral alignment tool having (i) a femoral alignment arm rotatably coupled to the shaft, (ii) a hip-knee-ankle arm segment located superior to the shaft, (iii) a hip-knee-ankle pointer located superior to the shaft, and (iv) a hip-knee-ankle angle gauge attached to the hip-knee-ankle arm segment, and
a tibial alignment tool extending inferiorly away from the shaft, the tibial alignment tool having (i) a tibial alignment arm rotatably coupled to the shaft, (ii) a varus/valgus arm segment located inferior to the shaft, (iii) a varus/valgus pointer located inferior to the shaft, and (iv) a varus/valgus angle gauge attached to the varus/valgus arm segment, wherein one of the varus/valgus pointer and the varus/valgus angle gauge is in a fixed orientation relative to the elongated base and the other of the varus/valgus pointer and the varus/valgus angle gauge is configured to rotate with the tibial alignment arm,
wherein one of the hip-knee-ankle pointer and the hip-knee-ankle angle gauge is in a fixed orientation relative to the tibial alignment arm and configured to rotate with the tibial alignment arm, and wherein the other of the hip-knee-ankle pointer and the hip-knee-ankle angle gauge is configured to rotate with the femoral alignment arm.

2. The orthopaedic surgical instrument assembly of claim 1, wherein:
the elongated base comprises a spacer block configured to be positioned between a resected femur and a resected tibia of the patient, the spacer block having a flat superior surface and a flat inferior surface, and
the shaft extends anteriorly away from the spacer block.

3. The orthopaedic surgical instrument assembly of claim 2, wherein a longitudinal axis of the varus/valgus pointer is orthogonal to the flat superior surface of the spacer block.

4. The orthopaedic surgical instrument assembly of claim 1, further comprising:
a femoral alignment rod extending superiorly away from a superior end of the femoral alignment arm, the femoral alignment rod being extendable and retractable relative to the superior end of the femoral alignment arm, and
a tibial alignment rod extending inferiorly away from an inferior end of the tibial alignment arm, the tibial alignment rod being extendable and retractable relative to the interior end of the tibial alignment arm.

5. The orthopaedic surgical instrument assembly of claim 1, wherein:
the femoral alignment arm has a longitudinal axis,
the hip-knee-ankle arm segment has a longitudinal axis,
the longitudinal axis of the femoral alignment arm is colinear with the longitudinal axis of the hip-ankle-knee arm segment.

6. The orthopaedic surgical instrument assembly of claim 1, wherein:
the varus/valgus angle gauge includes a plurality of angle indicators each of which corresponds to a different varus/valgus angle, and
rotation of the tibial alignment arm causes the varus/valgus angle gauge to be moved relative to the varus/valgus pointer such that the varus/valgus pointer aligns with one of the plurality of angle indicators.

7. The orthopaedic surgical instrument assembly of claim 1, wherein:
the femoral alignment arm and the tibial alignment arm form an anteroposterior angle, and
the anteroposterior angle is between 155 degrees to 175 degrees.

8. The orthopaedic surgical instrument assembly of claim 1, wherein:
the hip-knee-ankle angle gauge includes a plurality of angle indicators each of which corresponds to a different hip-knee-ankle angle, and
rotation of the tibial alignment arm causes the hip-knee-ankle angle gauge to be moved relative to the hip-knee-ankle pointer such that the hip-knee-ankle pointer aligns with one of the plurality of angle indicators.

9. The orthopaedic surgical instrument assembly of claim 8, wherein rotation of the femoral alignment arm causes the hip-knee-ankle pointer to be moved relative to the hip-knee-ankle angle gauge such that the hip-knee-ankle pointer aligns with one of the plurality of angle indicators.

10. The orthopaedic surgical instrument assembly of claim 1, wherein:
the femoral alignment arm and the tibial alignment arm rotate relative to one another about a hub,
the hub has a cavity formed therein that is sized and shaped to receive the shaft, and
a spring is positioned in the hub to selectively retain the shaft therein.

11. A method of surgically preparing a patient’s femur and tibia during an orthopaedic surgical knee procedure, comprising:
resecting a distal end of the patient’s femur and a proximal end of the patient’s tibia,
inserting a spacer block of a boundary assessment instrument between the resected distal end of the patient’s femur and the resected proximal end of the patient’s tibia,
aligning a tibial alignment rod of the boundary assessment instrument with an ankle of the patient,
determining, with the tibial alignment rod of the boundary assessment instrument aligned with the patient’s ankle, a varus/valgus angle from a varus/valgus angle gauge of the boundary assessment instrument,
aligning a femoral alignment rod of the boundary assessment instrument with a hip of the patient, and
determining, with the femoral alignment rod of the boundary assessment instrument aligned with the patient’s hip, a hip-knee-ankle angle from a hip-knee-ankle angle gauge of the boundary assessment instrument.

12. The method of claim 11, further comprising positioning the patient’s femur and tibia in extension prior to insertion of the spacer block.

13. The method of claim 11, wherein the tibial alignment rod is aligned with the patient’s ankle during alignment of the femoral alignment rod with the patient’s hip.

14. The method of claim 11, wherein:
aligning the tibial alignment rod comprises rotating the tibial alignment rod relative to the spacer block so as to align the tibial alignment rod with the patient’s ankle, and
aligning the femoral alignment rod comprises rotating the femoral alignment rod relative to the spacer block so as to align the femoral alignment rod with the patient’s hip.

15. The method of claim 14, wherein rotating the femoral alignment rod relative to the spacer block comprises rotating the femoral alignment rod relative to both the spacer block and the tibial alignment rod so as to align the femoral alignment rod with the patient’s hip.

16. The method of claim 11, wherein:
the boundary assessment instrument comprises a varus/valgus pointer, and
aligning the tibial alignment rod comprises moving the varus/valgus angle gauge relative to the varus/valgus pointer.

17. The method of claim 16, wherein:
the varus/valgus angle gauge includes a plurality of angle indicators each of which corresponds to a different varus/valgus angle, and
aligning the tibial alignment rod comprises moving the varus/valgus angle gauge relative to the varus/valgus pointer such that the varus/valgus pointer aligns with one of the plurality of angle indicators.

18. The method of claim 11, wherein:
the boundary assessment instrument comprises a varus/valgus pointer,
the varus/valgus angle gauge is secured to the tibial alignment rod, and
aligning the tibial alignment rod comprises moving the varus/valgus angle gauge relative to the varus/valgus pointer.

19. The method of claim 11, wherein:
the boundary assessment instrument comprises a hip-knee-ankle pointer,
aligning the tibial alignment rod includes rotating one of the hip-knee-ankle angle gauge and the hip-knee-ankle pointer with the tibial alignment rod, and
aligning the femoral alignment rod comprises moving the other of the hip-knee-ankle angle gauge and the hip-knee-ankle pointer.

20. The method of claim 19, wherein:
the hip-knee-ankle angle gauge includes a plurality of angle indicators each of which corresponds to a different hip-knee-ankle angle, and
aligning the femoral alignment rod comprises moving one of the hip-knee-ankle angle gauge and the hip-knee-ankle pointer relative to the other of the hip-knee-ankle angle gauge and the hip-knee-ankle pointer such that the hip-knee-ankle pointer aligns with one of the plurality of angle indicators.
